(19) United States
(12) Patent Application Publication
(10) Pub. No.: US 2017/0348330 A1
(43) Pub. Date: Dec. 7, 2017

(54) ANTIVIRAL COMPOUND AND ANALOGUES THEREOF FOR TREATMENT OR PREVENTION OF FLAVIVIRUS DENGUE/ZIKA INFECTIONS

(71) Applicant: Canopus BioPharma Incorporated, Studio City, CA (US)

(72) Inventor: Patrick Prendergast, Dublin (IE)

(21) Appl. No.: 15/171,104

(22) Filed: Jun. 2, 2016

(51) Int. Cl. A61K 31/585; A61K 9/70; A61K 9/00; A61K 45/06; A61K 31/566

(52) U.S. Cl. CPC A61K 31/585; A61K 45/06; A61K 31/566; A61K 9/0014; A61K 9/0053; A61K 9/0019; A61K 9/7023

(57) ABSTRACT

The present invention provides for an antiviral compound and analogues thereof for treatment or prevention of Flavivirus Dengue/Zika infections. In general the antiviral compound includes Eplerenone [pregn-4-ene-7, 21-dicarboxylic acid, 9,11-epoxy-17-hydroxy-3-oxo, γ-lactone, methyl ester (7α, 11α, 17α)] and its metabolites. Methods presented include treating Flavivirus infections, such as Zika virus, dengue virus, yellow fever virus and tick-borne encephalitis virus and Japanese encephalitis virus by administering the compound Eplerenone in therapeutically effective amounts and specific formulations as disclosed.

### FIELD OF THE INVENTION

This invention relates to the use of Eplerenone and its analogues, as well as compositions containing the same, for the treatment and/or prophylaxis of Flavivirus diseases associated with such viruses as Dengue fever, Zika virus Yellow fever, West Nile, St. Louis encephalitis, Hepatitis C, Murray Valley encephalitis, and Japanese encephalitis.

### BACKGROUND OF THE INVENTION

Dengue fever (DF) is an acute febrile disease caused by one of four closely related virus serotypes (DEN-1, DEN-2, DEN-3, and DEN-4). Dengue fever is classified based on its clinical characteristics into classical dengue fever, or the more severe forms, dengue hemorrhagic fever syndrome (DHF), and dengue shock syndrome (DSS). Recovery from infection from one serotype produces life-long immunity to that particular serotype, but provides only short-lived and limited protection against any of the other serotypes.

Dengue is a member of the Flaviviridae family which are enveloped, positive-sense RNA viruses whose human pathogens also include West Nile virus (WNV), yellow fever virus (YFV), Japanese encephalitis virus (JEV), and tick-borne encephalitis virus (TBEV) among others. Dengue transmission is via the bite of an infected Aedes aegypti mosquito which is found in tropical and sub-tropical regions around the world.

Each year regional epidemics of dengue cause significant morbidity and mortality, social disruption and substantial economic burden on the societies affected both in terms of hospitalization and mosquito control. Dengue is considered by the World Health Organization (WHO) to be the most important arthropod-borne viral disease with an estimated 50 million cases of dengue infection, including 500,000 DHF cases and 24,000 deaths worldwide each year (37, 38). WHO estimates that forty percent of the world's population (2.5 billion people) are at risk for DF, DHF, and DSS. Dengue is also a NIAID Category A pathogen and in terms of bio-defence, represents a significant threat to United States troops overseas. Dengue is an emerging threat to North America with a dramatic increase in severe disease in the past 25 years including major epidemics in Cuba and Venezuela, and outbreaks in Texas and Hawaii. Failure to control the mosquito vector and increases in long-distance travel have contributed to the increase and spread of dengue disease.

The characteristics of dengue as a viral hemorrhagic fever virus (arthropod-borne, widely spread, and capable of inducing a great amount of cellular damage and eliciting an immune response that can result in severe hemorrhage, shock, and death) makes this virus a unique threat to deployed military personnel around the world as well as to travellers to tropical regions. Preparedness for both biodefense and for the public health challenges posed by dengue will require the development of new vaccines and potent antiviral therapeutics with known side-effect profile.

Dengue causes several illnesses with increasing severity being determined in part by prior infection with a different serotype of the virus. Classic dengue fever (DF) begins 3-8 days after the bite of an infected mosquito and is characterized by sudden onset of fever, headache, back pain, joint pain, a measles-like rash, and nausea and vomiting. DF is frequently referred to as “breakbone” fever due to these symptoms. The disease usually resolves after two weeks but a prolonged recovery with weakness and depression is common. The more severe form of the disease, dengue hemorrhagic fever (DHF) has a similar onset and early phase of illness as dengue fever. However, shortly after onset the disease is characterized by high fever, enlargement of the liver, and hemorrhagic phenomena such as bleeding from the nose, mouth, and internal organs due to vascular permeability (38). In dengue shock syndrome (DSS) circulatory failure and hypovolaemic shock resulting from plasma leakage occur and can lead to death in 12-24 hours without plasma replacement (38). The case fatality rate of DHF/DSS can be as high as 20% without treatment. DHF has become a leading cause of hospitalization and death among children in many countries with an estimated 500,000 cases requiring hospitalization each year and a case fatality rate of about 5%.

The pathogenesis of DHF/DSS is still being studied but is thought to be due in part to an enhancement of virus replication in macrophages by heterotypic antibodies, termed antibody-dependent enhancement (ADE).

During a secondary infection, with a different serotype of dengue virus, cross-reactive antibodies that are not neutralizing form virus-antibody complexes that are taken into monocytes and Langerhans cells (dendritic cells) and increase the number of infected cells. This leads to the activation of cytotoxic lymphocytes which can result in plasma leakage and the hemorrhagic features characteristic of DHF and DSS. This antibody-dependent enhancement of infection is one reason why the development of a successful vaccine has proven to be so difficult. Although less frequent, DHF/DSS can occur after primary infection, so virus virulence and immune activation are also believed to contribute to the pathogenesis of the disease.

Dengue is endemic in more than 100 countries in Africa, the Americas, the Eastern Mediterranean, South-east Asia and the Western Pacific. During epidemics, attack rates can be as high as 80-90% of the susceptible population. All four serotypes of the virus are emerging worldwide, increasing the number of cases of the disease as well as the number of explosive outbreaks. In 2002 for example, there were 1,015,420 reported cases of dengue in the Americas alone with 14,374 cases of DHF, which is more than three times the number of dengue cases reported in the Americas in 1995.

Current management of dengue virus-related disease relies solely on vector control. There are no approved anti-viral drugs or vaccines for the treatment or prevention of dengue. Ribavirin, a guanosine analogue, has been shown to be effective against a range of RNA virus infections and works against dengue in tissue culture by inhibiting the dengue 2′-O-methyltransferase NS5 domain. However, Ribavirin did not show protection against dengue in a mouse model or a rhesus monkey model, instead it induced anemia and thrombocytosis. While there are no currently available approved vaccines, multivalent dengue vaccines have shown some limited potential in humans. However, vaccine development is difficult due to the presence of four distinct serotypes of the dengue virus which each cause disease.

Vaccine development also faces the challenge of ADE where unequal protection against the different strains of the virus could actually increase the risk of more serious disease. Therefore there is a need for antiviral drugs that target all of the serotypes of dengue. An antiviral drug administered early during dengue infection that inhibits viral replication would prevent the high viral load associated with DHF and be an attractive strategy in the treatment and prevention of disease. An antiviral drug that inhibits viral replication could be administered prior to travel to a dengue endemic region to prevent acquisition of disease, or for those that have previously been exposed to dengue, could prevent infection by another serotype of dengue virus and decrease the chance of life-threatening DHF and DSS occurring.

Having an antiviral drug would also aid vaccine development by having a tool at hand to treat complications that may arise due to unequal immune protection against the different serotypes. Although a successful vaccine could be a critical component of an effective bio-defence the typical delay to onset of immunity, potential side-effects, cost, and logistics associated with large-scale civilian vaccinations against a low-threat risk agent suggest that a comprehensive bio-defence include a separate rapid response element. Thus, there remains an urgent need to develop a safe and effective antiviral to protect against Flavivirus infection.

One such virus, the Zika virus is a member of the virus family Flaviviridae and the genus Flavivirus. This virus is spread by daytime-active Aedes mosquitoes, such as A. Aegypti and A. albopictus. The virus name is derived from the Zika Forest of Uganda, where the virus was first isolated in 1947. Zika virus is related to dengue virus, yellow fever, Japanese encephalitis, and West Nile viruses. The infection, known as Zika fever, often causes no symptoms or only mild symptoms, similar to a mild form of dengue fever. It is treated by rest. Generally, it had been known to occur only within a narrow equatorial belt from Africa to Asia. The virus spread eastward across the Pacific Ocean in 2013-2014 Zika virus outbreaks in Oceania to French Polynesia, New Caledonia, the Cook Islands, and Easter Island, and in 2015 to Mexico, Central America, the Caribbean, and South America, where the Zika virus outbreak has now reached pandemic levels. As of 2016, the illness cannot be prevented by medications or vaccines. Zika may spread from a pregnant woman to her baby in the womb. This may result in microcephaly and other severe brain problems. Zika infections in adults can result in Guillain-Barré syndrome.

### SUMMARY OF THE INVENTION

Accordingly, the present disclosure provides a compound and methods of treatment for use in the prophylaxis and therapy of a Flavivirus infection or a complication thereof. The treatment includes the administration of an active agent, herein described herein in the formulation of Formula I and direct anti-viral evidence via administration of Eplerenone, a prescription drug approved for administration as an adjunct in the management of chronic heart failure.

Eplerenone is a potassium sparing diuretic medication. The present invention provides a pharmaceutical composition comprising a pharmaceutically acceptable carrier and a compound having the following general formula I or a pharmaceutically acceptable salt thereof:

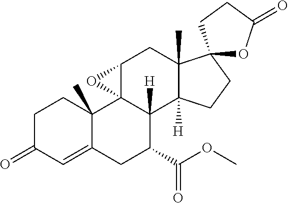

### Formula 1—Eplerenone

In some implementations, a method for the treatment a Flavivirus infection or prophylaxis of the Flavivirus infection, or disease associated with a Flavivirus infection is disclosed that includes administering to a patient an amount of Eplerenone (pregn-4-ene-7,21-dicarboxylic acid, 9,11-epoxy-17-hydroxy-3-oxo, γ-lactone, methyl ester (7α, 11α, 17α) effective to contain an increase in the Flavivirus infection. In addition, intravenous fluid may be administered to the patient at one or both of: simultaneously with the administering to the patient an amount of Eplerenone or following the administration of an amount of Eplerenone to the patient.
In another aspect, it may be determined that the patient has a Flavivirus infection prior to the administration to the patient of the Eplerenone (pregn-4-ene-7,21-dicarboxylic acid, 9,11-epoxy-17-hydroxy-3-oxo, γ-lactone, methyl ester (7α, 11α, 17α) and also determined that the patient has contained the Flavivirus infection after the administration of pregn-4-ene-7,21-dicarboxylic acid, 9,11-epoxy-17-hydroxy-3-oxo, γ-lactone, methyl ester (7α, 11α, 17α).
In various implementations, the pregn-4-ene-7,21-dicarboxylic acid, 9,11-epoxy-17-hydroxy-3-oxo, γ-lactone, methyl ester (7α, 11α, 17α) may be administered via one or more of: intra venous introduction; an oral ingestion and transdermal patch.
In another aspect, the pregn-4-ene-7,21-dicarboxylic acid, 9,11-epoxy-17-hydroxy-3-oxo, γ-lactone, methyl ester (7α, 11α, 17α) may have demonstrated an antiviral effect against the Dengue virus covering each of strains DEN-1, DEN-2, DEN-3 and DEN-4. The Dengue fever may be selected from a group including one or more of: classical dengue fever, dengue hemorrhagic fever syndrome, and dengue shock syndrome.
In still another aspect at least one agent selected from a group including: an antiviral agent, a vaccine, interferon and a therapeutic compound may be co-administered.
The pregn-4-ene-7,21-dicarboxylic acid, 9,11-epoxy-17-hydroxy-3-oxo, γ-lactone, methyl ester (7α, 11α, 17α) may be administered systemically via introduction into the bloodstream of the patient.
The pregn-4-ene-7,21-dicarboxylic acid, 9,11-epoxy-17-hydroxy-3-oxo, γ-lactone, methyl ester (7α, 11α, 17α) may also have demonstrated antiviral effect against the Zika virus and be administered to treat a Zika virus infection in a human.
Some implementations include methods of treating or preventing a Zika virus infection of a fetus by administering an antiviral dose of Eplerenone to a pregnant mother of the fetus. The pregn-4-ene-7,21-dicarboxylic acid, 9,11-epoxy-17-hydroxy-3-oxo, γ-lactone, methyl ester (7α, 11α, 17α) may be administered to the mother of the fetus via one or more of: transdermal patch; intra venous introduction and oral introduction. Intravenous fluid may also be provided to the mother of the fetus in correlation with the treatment involving administration of the pregn-4-ene-7,21-dicarboxylic acid, 9,11-epoxy-17-hydroxy-3-oxo, γ-lactone, methyl ester (7α, 11α, 17α).

Other objects and advantages of the present invention will become apparent from the following description including the antiviral results, accompanying diagrams and claims. For example, the compounds and methods presented herein may be used in the treatment of additional virus strains, such as Nimodipine, Felodipine and Amlodipine.

### BRIEF DESCRIPTION OF THE DRAWINGS

The accompanying drawings, that are incorporated in and constitute a part of this specification, illustrate several embodiments of the disclosure and, together with the description, serve to explain the principles of the disclosure:

FIGS. 1 through 4 illustrate anti-viral activity in chart form demonstrating effectiveness of administration of active agents described herein.

FIGS. 5 and 6 illustrate Eplerenone Cmax concentrations achieved in normal subjects.

FIG. 7 illustrates a flow chart of method steps that may be implemented in some embodiments of the present invention.

### DETAILED DESCRIPTION OF THE INVENTION

The present invention includes a treatment or prophylaxis of a Flavivirus infection or disease associated therewith. According to the present invention, a therapeutically effective amount of Formula 1 is administered to a mammal. In general, the mammal will be a human being or a domesticated animal, although other veterinary uses are within the scope of the present invention.

The compounds of the invention are of the general Formula I:

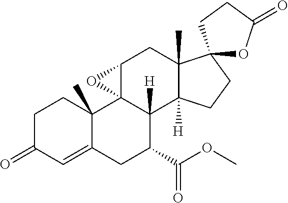

In some specific embodiments, the mammal is a human and the viral infection is a Flavivirus infection. The Flavivirus virus may be selected, for example, from a group including Dengue virus, Zika virus, West Nile virus, Yellow fever virus, Japanese encephalitis virus, and tick-borne encephalitis virus. Preferably, the Flavivirus is a Dengue virus selected from the group including DEN-1, DEN-2, DEN-3, and DEN-4, or a Zika viral infection.

The viral infection may be associated with a condition selected from a group including Dengue fever, Zika virus Yellow fever, West Nile, St. Louis encephalitis, Hepatitis C, Murray Valley encephalitis, and Japanese encephalitis. Dengue fever may be selected from a group including classical dengue fever, dengue hemorrhagic fever syndrome, and dengue shock syndrome.

The method of the present invention may also include co-administration of: a) other antivirals such as Ribavirin or cidofovir; b) vaccines; and/or c) interferons or pegylated interferons. (D) Drospirenone or Mifepristone to prevent viral haemorrhagic effects and hypovolaemic shock as outlined in another patent.

### Definitions

In accordance with this detailed description, the following abbreviations and definitions apply. It must be noted that as used herein, the singular forms “a,” “an,” and “the” include plural referents unless the context clearly dictates otherwise.

Where a range of values is provided, it is understood that each intervening value is encompassed. The upper and lower limits of these smaller ranges may independently be included in the smaller, subject to any specifically-excluded limit in the stated range. Where the stated range includes one or both of the limits, ranges excluding either both of those included limits are also included in the invention. Also contemplated are any values that fall within the cited ranges.

Unless defined otherwise, all technical and scientific terms used herein have the same meaning as commonly understood by one of ordinary skill in the art. Any methods and materials similar or equivalent to those described herein can also be used in practice or testing.

As used herein “administered to a patient” means entered into a patient's blood stream according to the methods and means described herein.

As used herein “efficacy” in the context of a chronic dosage regime refers to the effectiveness of a particular treatment regime. Efficacy can be measured based on change of the course of the disease in response to an agent.

As used herein “patient” or “subject” is meant to include a mammal. A “mammal,” for purposes of treatment, refers to any animal classified as a mammal, including but not limited to, humans, experimental animals including rats, mice, and guinea pigs, domestic and farm animals, and zoo, sports, or pet animals, such as dogs, horses, cats, cows, camels and the like.

As used herein the term “success” rein in the context of a chronic treatment regime refers to the effectiveness of a particular treatment regime. This includes a balance of efficacy, toxicity (e.g., side effects and patient tolerance of a formulation or dosage unit), patient compliance, and the like. For a chronic administration regime to be considered “successful” it must balance different aspects of patient care and efficacy to produce a favourable patient outcome.

As used herein “treating,” “treatment,” and the like are used herein to refer to obtaining a desired pharmacological and physiological effect. The effect may be prophylactic in terms of preventing or partially preventing a disease, symptom, or condition thereof and/or may be therapeutic in terms of a partial or complete cure of a disease, condition, symptom, or adverse effect attributed to the disease. The term “treatment,” as used herein, covers any treatment of a disease in a mammal, such as a human, and includes: (a) preventing the disease from occurring in a subject which may be predisposed to the disease but has not yet been diagnosed as having it, i.e., causing the clinical symptoms of the disease not to develop in a subject that may be predisposed to the disease but does not yet experience or display symptoms of the disease; (b) inhibiting the disease, i.e., arresting or reducing the development of the disease or its clinical symptoms; and (c) relieving the disease, i.e., causing regression of the disease and/or its symptoms or conditions. Treating a patient's suffering from disease related to pathological inflammation is contemplated. Preventing, inhibiting, or relieving adverse effects attributed to pathological inflammation over long periods of time and/or are such caused by the physiological responses to inappropriate inflammation present in a biological system over long periods of time are also contemplated.

As used herein “Pharmaceutically-acceptable carrier” means a carrier that is useful in preparing a pharmaceutical composition or formulation that is generally safe, non-toxic, and neither biologically nor otherwise undesirable, and includes a carrier that is acceptable for veterinary use as well as human pharmaceutical use. A pharmaceutically-acceptable carrier or excipient includes one or more than one of such carriers.

As used herein “Pharmaceutically-acceptable cation” refers to the cation of a pharmaceutically-acceptable salt. “Pharmaceutically-acceptable salt” refers to salts which retain the biological effectiveness and properties of compounds which are not biologically or otherwise undesirable. Pharmaceutically-acceptable salts refer to pharmaceutically-acceptable salts of the compounds, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

Pharmaceutically-acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group including alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. It should also be understood that other carboxylic acid derivatives would be useful, for example, carboxylic acid amides, including carboxamides, lower alkyl carboxamides, dialkyl carboxamides, and the like.

Pharmaceutically-acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

A compound may act as a pro-drug. Pro-drug means any compound which releases an active parent drug in vivo when such pro-drug is administered to a mammalian subject. Pro-drugs are prepared by modifying functional groups present in such a way that the modifications may be cleaved in vivo to release the parent compound. Prodrugs include compounds wherein a hydroxy, amino, or sulfhydryl group is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), carbamates (e.g., N,N-dimethylamino-carbonyl) of hydroxy functional groups, and the like.

“Treating” or “treatment” of a disease includes: (1) preventing the disease, i.e. causing the clinical symptoms of the disease not to develop in a mammal that may be exposed to or predisposed to the disease but does not yet experience or display symptoms of the disease, (2) inhibiting the disease, i.e., arresting or reducing the development of the disease or its clinical symptoms, or (3) relieving the disease, i.e., causing regression of the disease or its clinical symptoms.

A “therapeutically-effective amount” means the amount of a compound that, when administered to a mammal for treating a disease, is sufficient to effect such treatment for the disease. The “therapeutically-effective amount” will vary depending on the compound, the disease, and its severity and the age, weight, etc., of the mammal to be treated.

### Pharmaceutical Formulations of the Compounds

In general, compounds will be administered in a therapeutically-effective amount by any of the accepted modes of administration for these compounds. The compounds can be administered by a variety of routes, including, but not limited to, oral, parenteral (e.g., subcutaneous, subdural, intravenous, intramuscular, intrathecal, intraperitoneal, intracerebral, intra-arterial, or intralesional routes of administration), topical, intranasal, localized (e.g., surgical application or surgical suppository), rectal, and pulmonary (e.g., aerosols, inhalation, or powder). Accordingly, these compounds are effective as both injectable and oral compositions. The compounds can be administered continuously by infusion or by bolus injection. The actual amount of the compound, i.e., the active ingredient, will depend on a number of factors, such as the severity of the disease, i.e., the condition or disease to be treated, age, and relative health of the subject, the potency of the compound used, the route and form of administration, and other factors.

Toxicity and therapeutic efficacy of such compounds can be determined by standard pharmaceutical procedures in cell cultures or experimental animals, e.g., for determining the LD.sub.50 (the dose lethal to 50% of the population) and the ED.sub.50 (the dose therapeutically effective in 50% of the population). The dose ratio between toxic and therapeutic effects is the therapeutic index and it can be expressed as the ratio LD.sub.50/ED.sub.50.

The data obtained from the cell culture assays and animal studies can be used in formulating a range of dosage for use in humans. The dosage of such compounds lies within a range of circulating concentrations that include the ED.sub.50 with little or no toxicity. The dosage may vary within this range depending upon the dosage form employed and the route of administration utilized. For any compound used, the therapeutically-effective dose can be estimated initially from cell culture assays. A dose may be formulated in animal models to achieve a circulating plasma concentration range which includes the IC.sub.50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The amount of a pharmaceutical composition administered to the patient will vary depending upon what is being administered, the purpose of the administration, such as prophylaxis or therapy, the state of the patient, the manner of administration, and the like. In therapeutic applications, compositions are administered to a patient already suffering from a disease in an amount sufficient to cure or at least partially arrest the symptoms of the disease and its complications. An amount adequate to accomplish this is defined as “therapeutically-effective dose.” Amounts effective for this use will depend on the disease condition being treated as well as by the judgment of the attending clinician depending upon factors such as the severity of the inflammation, the age, weight, and general condition of the patient, and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. It will be understood that use of certain of the foregoing excipients, carriers, or stabilizers will result in the formation of pharmaceutical salts.

The active compound is effective over a wide dosage range and is generally administered in a pharmaceutically- or therapeutically-effective amount. The therapeutic dosage of the compounds will vary according to, for example, the particular use for which the treatment is made, the manner of administration of the compound, the health and condition of the patient, and the judgment of the prescribing physician.

For example, for intravenous administration, in some preferred embodiments, therapeutically-effective amount will be in the range of about 0.5 mg to about 100 mg per kilogram body weight. Effective doses can be extrapolated from dose-response curves derived from in vitro or animal model test systems. A clinician or other health care provider, administers the compound until a dosage is reached that achieves the desired effect. Other embodiments may include a therapeutically-effective amount in a dosage of between about 0.25 mg to 200 mg.

When employed as pharmaceuticals, the compounds are usually administered in the form of pharmaceutical compositions. Pharmaceutical compositions contain as the active ingredient one or more of the compounds above, associated with one or more pharmaceutically-acceptable carriers or excipients. The excipient employed is typically one suitable for administration to human subjects or other mammals. In making the compositions, the active ingredient is usually mixed with an excipient, diluted by an excipient, or enclosed within a carrier which can be in the form of a capsule, sachet, paper or other container. When the excipient serves as a diluent, it can be a solid, semi-solid, or liquid material, which acts as a vehicle, carrier, or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g., about 40 mesh. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained, or delayed-release of the active ingredient after administration to the patient by employing procedures known in the art.

The quantity of active compound in the pharmaceutical composition and unit dosage form thereof may be varied or adjusted widely depending upon the particular application, the manner or introduction, the potency of the particular compound, and the desired concentration. The term “unit dosage forms” refers to physically-discrete units suitable as unitary dosages for human subjects and other mammals, each unit containing a predetermined quantity of active material calculated to produce the desired therapeutic effect, in association with a suitable pharmaceutical excipient.

Administration of therapeutic agents by intravenous formulation is well known in the pharmaceutical industry. An intravenous formulation should possess certain qualities aside from being just a composition in which the therapeutic agent is soluble. For example, the formulation should promote the overall stability of the active ingredient(s), also, the manufacture of the formulation should be cost-effective. All of these factors ultimately determine the overall success and usefulness of an intravenous formulation.

The compositions are preferably formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compounds are effective over a wide dosage range and are generally administered in a pharmaceutically effective amount. It will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient may be mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 2000 mg of the active ingredient.

The tablets or pills may be coated or otherwise compounded to provide a dosage form affording the advantage of prolonged action. For example, the tablet or pill can include an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions may be incorporated for administration orally or by injection include aqueous solutions suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation may include solutions and suspensions in pharmaceutically-acceptable, organic solvents, or mixtures thereof, and powders. The liquid or solid compositions may contain suitable pharmaceutically-acceptable excipients as described supra. Compositions in pharmaceutically-acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered from devices which deliver the formulation in an appropriate manner.

In some embodiments, the compounds may also be administered in a sustained release form. Suitable examples of sustained-release preparations include semipermeable matrices of solid hydrophobic polymers containing the compounds, which matrices are in the form of shaped articles, e.g., films, or microcapsules.

Examples of sustained-release matrices include polyesters, hydrogels (e.g., poly (2-hydroxyethyl-methacrylate) as described by Langer et al., J. Biomed. Mater. Res. 15: 167-277 (1981) and Langer, Chem. Tech. 12: 98-105 (1982) or poly(vinyl alcohol)), polylactides (U.S. Pat. No. 3,773,919), copolymers of L-glutamic acid and gamma ethyl-L-glutamate (Sidman et al., Biopolymers 22: 547-556, 1983), non-degradable ethylene-vinyl acetate (Langer et al., supra), degradable lactic acid-glycolic acid copolymers such as the LUPRON DEPOT™ (i.e., injectable microspheres composed of lactic acid-glycolic acid copolymer and leuprolide acetate), and poly-D-(−)-3-hydroxybutyric acid (EP 133,988).

The compounds may be administered in a sustained-release form, for example a depot injection, implant preparation, or osmotic pump, which can be formulated in such a manner as to permit a sustained-release of the active ingredient. Implants for sustained-release formulations are well-known in the art. Implants may be formulated as, including but not limited to, microspheres, slabs, with biodegradable or non-biodegradable polymers. For example, polymers of lactic acid and/or glycolic acid form an erodible polymer that is well-tolerated by the host.

Transdermal delivery devices (“patches”) may also be employed to administer the active agents described herein. Such transdermal patches may be used to provide continuous or discontinuous infusion of the compounds in controlled amounts. The construction and use of transdermal patches for the delivery of pharmaceutical agents is well known in the art. See, e.g., U.S. Pat. No. 5,023,252, issued Jun. 11, 1991, herein incorporated by reference. Such patches may be constructed for continuous, pulsatile, or on-demand delivery of pharmaceutical agents.

Direct or indirect placement techniques may be used when it is desirable or necessary to introduce the pharmaceutical composition to the brain. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472, which is herein incorporated by reference. Indirect techniques usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid-soluble and amenable to transportation across the blood-brain barrier. Alternatively, the delivery of hydrophilic drugs may be enhanced by intra-arterial infusion of hypertonic solutions which can transiently open the blood-brain barrier.

In order to enhance serum half-life, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka et al., U.S. Pat. Nos. 4,235,871, 4,501,728 and 4,837,028 each of which is incorporated herein by reference.

Pharmaceutical compositions are suitable for use in a variety of drug delivery systems. Suitable formulations for use in the present invention are found in Remington's Pharmaceutical Sciences, Mace Publishing Company, Philadelphia, Pa., 17th ed. (1985).

The provided compounds and pharmaceutical compositions show biological activity in treating and preventing viral infections and associated diseases, and, accordingly, have utility in treating viral infections and associated diseases, such as Hemorrhagic fever viruses, in mammals including humans.

Hemorrhagic fever viruses (HFVs) are RNA viruses that cause a variety of disease syndromes with similar clinical characteristics. HFVs that are of concern as potential biological weapons include but are not limited to: Arenaviridae (Junin, Machupo, Guanarito, Sabia, and Lassa), Filoviridae (Ebola and Marburg viruses), Flaviviridae (yellow fever, Omsk hemorrhagic fever and Kyasanur Forest disease viruses), and Bunyaviridae (Rift Valley fever and Crimean-Congo hemorrhagic fever). The naturally-occurring arenaviruses and potential engineered arenaviruses are included in the Category A Pathogen list according to the Centers for Disease Control and Prevention as being among those agents that have greatest potential for mass casualties.

Risk factors include: travel to South America, Caribbean, Africa or Asia, handling of animal carcasses, contact with infected animals or people, and/or arthropod bites. Arenaviruses are highly infectious after direct contact with infected blood and/or bodily secretions. Humans usually become infected through contact with infected rodents, the bite of an infected arthropod, direct contact with animal carcasses, inhalation of infectious rodent excreta and/or injection of food contaminated with rodent excreta. The Tacaribe virus has been associated with bats. Airborne transmission of hemorrhagic fever is another mode. Person-to-person contact may also occur in some cases.

All of the hemorrhagic fevers exhibit similar clinical symptoms. However, in general the clinical manifestations are non-specific and variable. The incubation period is approximately 7-14 days. The onset is gradual with fever and malaise, tachypnea, relative bradycardia, hypotension, circulatory shock, conjunctival infection, pharyngitis, lymphadenopathy, encephalitis, myalgia, back pain, headache and dizziness.

### Example 1

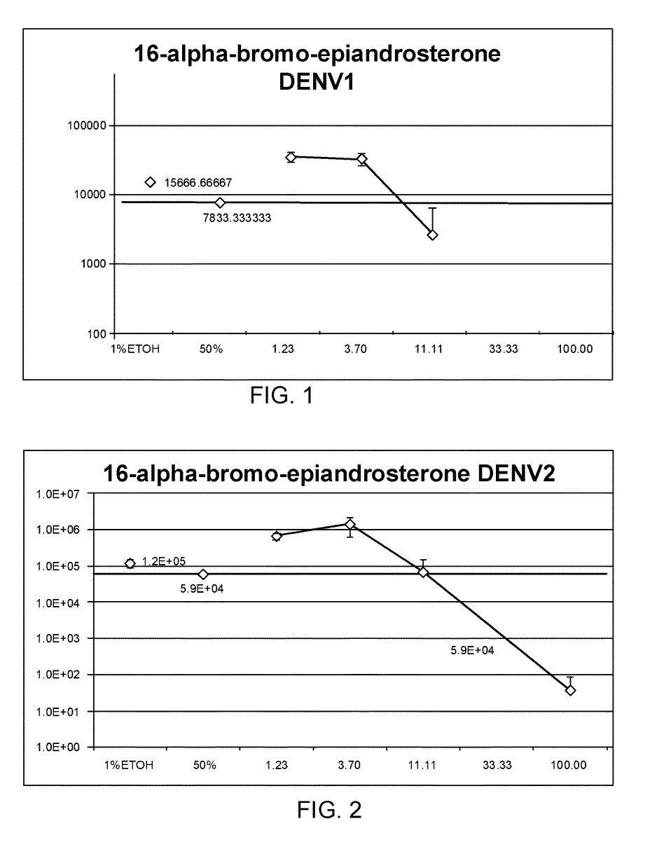

Referring now to FIGS. 1-4, illustrations of anti-viral activity are included in chart form to demonstrate the effectiveness of administration of the active agents described herein as well as the determined selectivity or specificity of compounds of the disclosed herein.

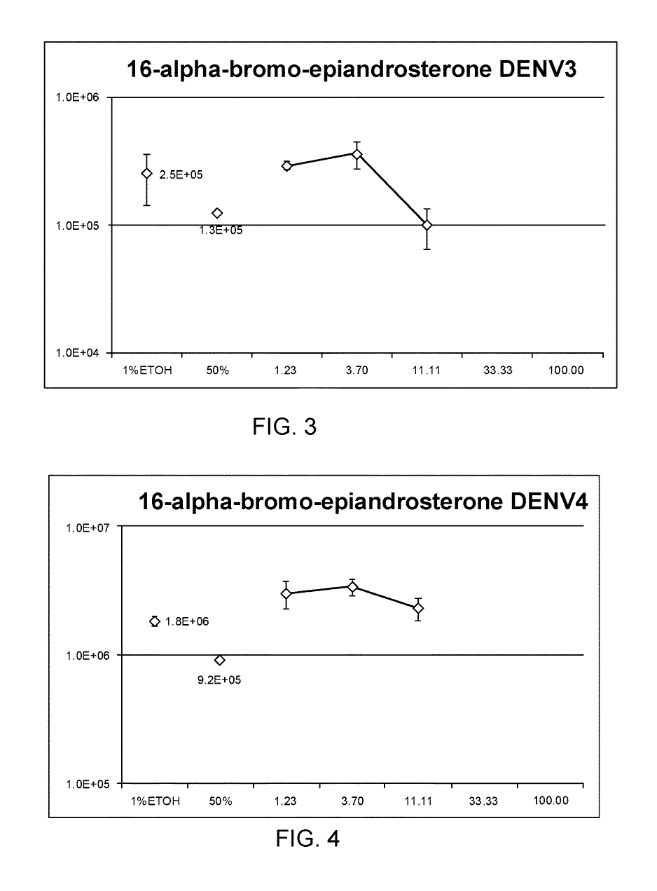

In general, those compounds with activity against dengue-2 at effective concentrations of less than 10 .mu.M as identified in FIGS. 1-4 and including: DENV1 in FIG. 1; DENV2 in FIG. 2; DENV3 in FIG. 3; and DENV4 in FIG. 1. Each of DENV1-4 were tested for activity against each serotype of dengue in a viral yield assay to generate EC50 values.

Eplerenone was also tested for more broad spectrum activity against other members of the Flaviviridae family including murine Flavivirus, as well as Bovine Viral Diarrhea Virus (BVDV), which is a Pestivirus. Since dengue virus is able to replicate in multiple cell lines and to ensure that the activity seen in vero cells is consistent, Eplerenone was also tested for its effective concentration in a viral yield assay against dengue-2 in C6/36 mosquito cells.

ZIKA Anti-Viral In-Vitro Testing and Antiviral Assay Report:

Samples tested: Nimodipine, Spironolactone, Felodipine, Amlodipine Besylate, Eplerenone

Controls: Medium with DMSO at 1% start concentration; Zika virus for infection at 0.1 MOI; VERO cells on VP medium (serum-free).

Procedure: Antivirals were diluted as information provided to stock concentrations of 10 mM in 100% DMSO. The first dilution for analysis resulted in a final concentration of 1% DMSO in the cell culture.

### Steps:

1—VERO cells in VP were inoculated in 200 μL/well at 5.0×10⁴ cells/mL in 96-well plates.
2—Incubated at 37° C. for 48 hours.
3—With 80% confluence the test was performed as follows: culture medium was changed for 150 μL of fresh VP in columns 2 to 10. Antivirals diluted at 100 μM in VP medium were placed in the column 1 (300 μL). Serial dilutions from 1 to 10 were performed by passing 150 μL of medium (1:2). Plates were incubated at 37° C. for 2 hours. Zika virus diluted in VP medium to the final infection ration desired of MOI 0.1 in 100 μL of VP was added to wells.
4—Plates were incubated for 72 hours and checked each 24 hours for cytotoxicity or cytopathic effect.
5—Plates were washed and stained with Naftol blue-black. Absorbance was measured at 450 nm.

### Results:

Mean absorbance of 36 non-infected wells (mock infection): 0.634
Mean absorbance of 42 infected wells (positive control): 0.382 (Standard Deviation SD: 0.0445)

### Legend:

DMSO cytotoxicity
Antiviral cytotoxicity
Primary Threshold for virus inhibition. Absorbance above 0.382

| | 100 μM | 50 μM | 25 μM | 12.5 μM | 6.25 μM | 3.12 μM | 1.56 μM | 0.78 μM | 0.39 μM | 0.19 μM |
|---|---|---|---|---|---|---|---|---|---|---|
| Nimodipine | 0.045 | 0.091 | 0.21 | 0.276 | 0.263 | 0.235 | 0.202 | 0.142 | 0.12 | 0.206 |
| | 0.043 | 0.113 | 0.224 | 0.282 | 0.363 | 0.468 | 0.387 | 0.293 | 0.375 | 0.321 |
| | 0.057 | 0.117 | 0.271 | 0.347 | 0.451 | 0.451 | 0.46 | 0.459 | 0.367 | 0.378 |
| | 0.047 | 0.092 | 0.217 | 0.305 | 0.395 | 0.415 | 0.397 | 0.396 | 0.326 | 0.354 |
| Spironolactone | 0.048 | 0.079 | 0.255 | 0.338 | 0.358 | 0.359 | 0.328 | 0.328 | 0.304 | 0.22 |
| | 0.046 | 0.082 | 0.257 | 0.342 | 0.307 | 0.317 | 0.318 | 0.23 | 0.225 | 0.2 |
| | 0.051 | 0.074 | 0.214 | 0.234 | 0.263 | 0.223 | 0.2 | 0.168 | 0.215 | 0.183 |
| | 0.069 | 0.047 | 0.076 | 0.092 | 0.097 | 0.06 | 0.069 | 0.063 | 0.068 | 0.095 |
| Felodipine | 0.045 | 0.041 | 0.039 | 0.043 | 0.209 | 0.329 | 0.325 | 0.295 | 0.252 | 0.303 |
| | 0.045 | 0.039 | 0.044 | 0.049 | 0.368 | 0.47 | 0.474 | 0.45 | 0.466 | 0.457 |
| | 0.046 | 0.043 | 0.042 | 0.056 | 0.379 | 0.443 | 0.388 | 0.363 | 0.367 | 0.424 |
| | 0.051 | 0.051 | 0.057 | 0.056 | 0.341 | 0.425 | 0.399 | 0.401 | 0.402 | 0.401 |
| Amlodipine | 0.049 | 0.046 | 0.051 | 0.338 | 0.068 | 0.136 | 0.214 | 0.313 | 0.326 | 0.386 |
| Besylate | 0.046 | 0.045 | 0.048 | 0.248 | 0.07 | 0.139 | 0.192 | 0.273 | 0.285 | 0.316 |
| | 0.051 | 0.044 | 0.053 | 0.232 | 0.062 | 0.097 | 0.171 | 0.249 | 0.27 | 0.319 |
| | 0.044 | 0.036 | 0.05 | 0.129 | 0.061 | 0.104 | 0.16 | 0.212 | 0.159 | 0.297 |
| Eplerenone | 0.116 | 0.217 | 0.29 | 0.334 | 0.376 | 0.4 | 0.369 | 0.433 | 0.408 | 0.449 |
| | 0.155 | 0.238 | 0.288 | 0.333 | 0.448 | 0.423 | 0.466 | 0.434 | 0.484 | 0.441 |
| | 0.103 | 0.209 | 0.291 | 0.373 | 0.411 | 0.453 | 0.453 | 0.432 | 0.437 | 0.468 |
| | 0.107 | 0.182 | 0.271 | 0.341 | 0.397 | 0.441 | 0.443 | 0.428 | 0.414 | 0.469 |
| Antiviral | 0.152 | 0.199 | 0.477 | 0.452 | 0.366 | 0.423 | 0.422 | 0.451 | 0.447 | 0.397 |
| Butantan | 0.172 | 0.204 | 0.536 | 0.413 | 0.363 | 0.441 | 0.459 | 0.403 | 0.415 | 0.443 |
| Control* | 0.288 | 0.269 | 0.501 | 0.412 | 0.39 | 0.358 | 0.411 | 0.415 | 0.436 | 0.408 |
| | 0.292 | 0.26 | 0.311 | 0.275 | 0.385 | 0.329 | 0.303 | 0.28 | 0.279 | 0.333 |

Objective: Providing guidance on Eplerenone Pharmacokinetics in human and dosage needed to achieve maximum serum concentrations over a three day period to clear Zika virus in view of in-vitro study showing antiviral activity.

Conclusions:

Comparison of Eplerenone in-vitro concentrations (reported by KLM Biotechnology Ltd) with in-vivo Cmax concentrations following Eplerenone administration in humans:

Invitro anti-viral concentrations (KLM report):

In-vitro Eplerenone concentrations ranging from 0.19-3.12 μM showing anti-viral activity against Zika virus (Column#6-10; Page 2, KLM report) were comparable to Butantan control (which showed absorbance values above 0.382, the primary threshold for Zika virus inhibition).

Eplerenone concentration of 0.39 μM (column#9, KLM report) was associated with one absorbance value that is higher than 0.471 (an evident antiviral effect).

Clinical data: Eplerenone PK and concentrations reported in humans:

Peak plasma concentrations (Cmax) after therapeutic doses of oral Eplerenone (100 mg) can reach levels up to 1720 ng/ml (equivalent to 4.1 μM).

This is 10-times higher than the in-vitro concentrations reported in column#9. It also exceeds the whole range of concentrations (0.19-3.2 μM) reported in columns 6-10.

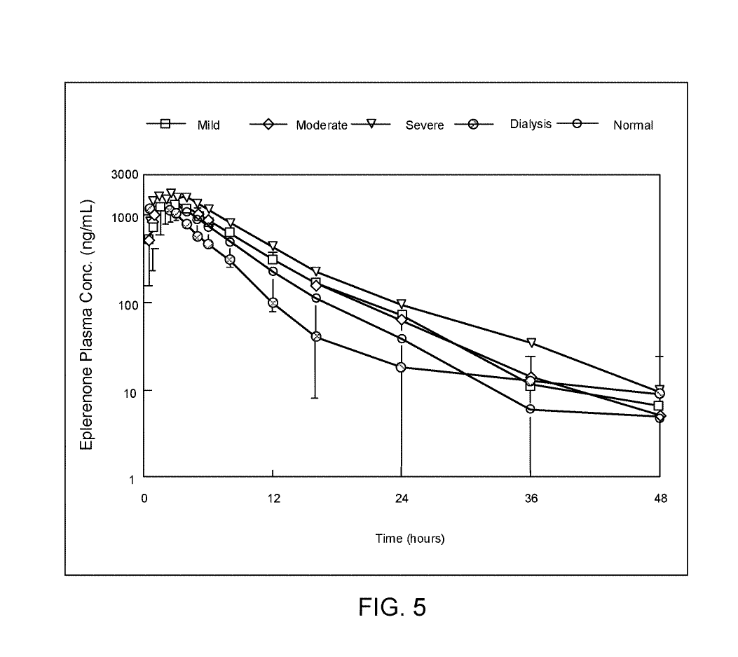

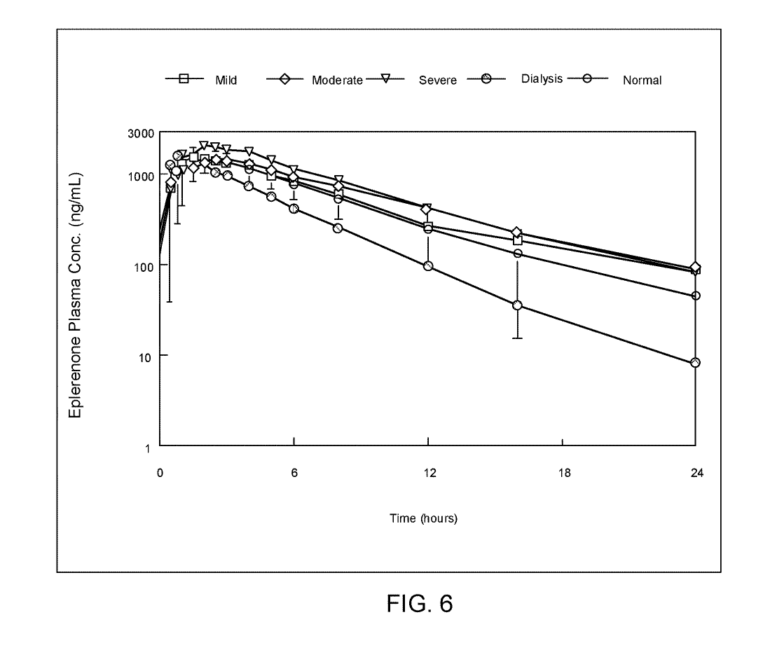

Referring now to FIG. 5 and FIG. 6, Eplerenone Cmax concentrations achieved in normal subjects is illustrated, as well as subjects with varying degrees of renal impairment after single and multiple doses of Eplerenone given orally.

The charts illustrate the feasibility of achieving anti-viral Cmax concentrations using Eplerenone in human and show Eplerenone plasma concentration vs. time curves. In healthy subjects who received single dose of 50 mg Eplerenone orally they show mean Cmax concentrations of 1173 ng/ml (equivalent to 2.8 M). Increasing the dose to 100 mg resulted in mean Cmax concentrations of 1720 ng/ml (equivalent to 4.1 μM).

In multiple dose studies, it is shown that Eplerenone steady state concentrations may be reached within 4 days. The measured steady state Cmax concentrations observed showed no significant accumulation when compared with single dose administration. Therefore, in order to achieve Cmax concentrations that are comparable to the in-vitro study, preferred embodiments include a daily dose of 100 mg oral Eplerenone (or more) over a 3-day period o clear Zika virus in a human. This preferred embodiment may be adjusted according to the specific conditions of the human subject.

Safety of Eplerenone Dose Suggested:

According to the Material Safety Data Sheets (MSDS) of Eplerenone.

Eplerenone is generally well tolerated. Two large, randomized, double-blind trials, showed that overall incidence of adverse events was similar between Eplerenone and placebo recipients.

The most common side-effect of Eplerenone is hypercalaemia, which may be addressed via close monitoring in subjects receiving treatment with diabetes mellitus, proteinuria or heart failure.

On some embodiments, serum potassium levels may be assessed prior to initiating Eplerenone therapy and within the first week. Eplerenone may be contraindicated in patients who have serum potassium levels >5.0 mmol/L at baseline.

Eplerenone is category B in pregnancy which means animal reproduction studies have failed to demonstrate a risk to the fetus and there are no adequate and well-controlled studies in pregnant women. In animal studies, the only noticeable risk of foetal abnormalities occurred when laboratory animals were given doses well over 1000 times the recommended daily dose of Eplerenone.

Accordingly, the present disclosure teaches that Eplerenone may be safely and effectively used throughout pregnancy at the doses recommended in treatments related to Flavivirus.

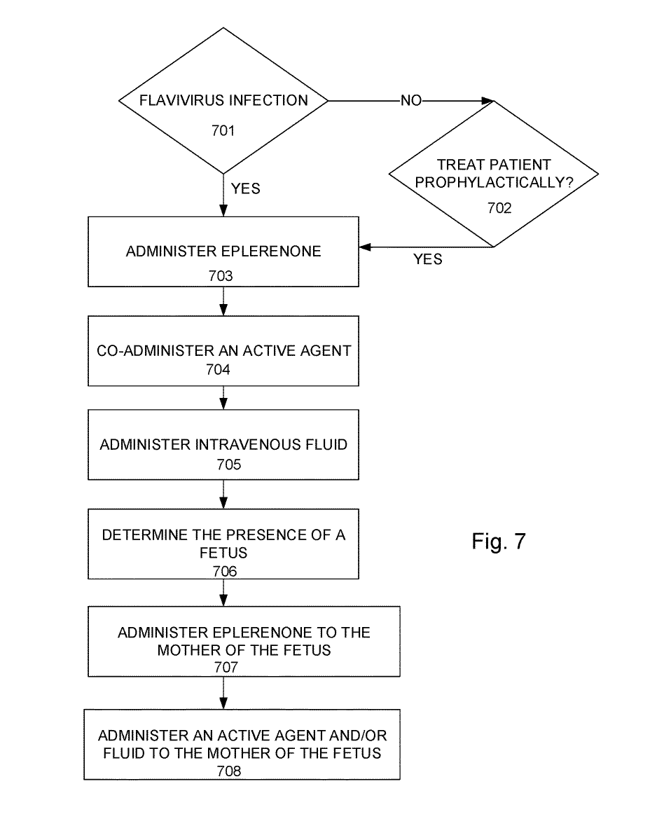

Referring now to FIG. 7, method steps are listed that may be implemented in addition to the steps described above for the treatment of a viral infection or prophylactically against a viral infection.

At method step 701, it may be determined if a viral infection is present in a patient, and in particular if a Flavivirus infection is present. At method step 702, if no infection is present, then it may be determined to treat the patient prophylactically. At method step 703 the treatment may include administration of Eplerenone as described herein.

At method step 704 in some embodiments, an active agent may be co-administered with the Eplerenone. Co-administration may be simultaneous and/or sequentially.

At method step 706, the presence of a fetus may be determined and at step 708 Eplerenone may be administered to the mother of the fetus in order to treat the fetus.

At method step 708, additional active agents and/or intravenous fluids may also be administered to the mother of the fetus.

A number of embodiments of the present disclosure have been described. While this specification contains many specific implementation details, there should not be construed as limitations on the scope of any disclosures or of what may be claimed, but rather as descriptions of features specific to particular embodiments of the present disclosure.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Thus, particular embodiments of the subject matter have been described. Other embodiments are within the scope of the following claims. In some cases, the actions recited in the claims can be performed in a different order and still achieve desirable results. Nevertheless, it will be understood that various modifications may be made without departing from the spirit and scope of the claimed disclosure.

What is claimed is:

1. A method for treatment of a Flavivirus infection or prophylaxis of the Flavivirus infection, or disease associated with the Flavivirus infection, the method comprising the step of:
administering to a patient an amount of pregn-4-ene-7,21-dicarboxylic acid, 9,11-epoxy-17-hydroxy-3-oxo, γ-lactone, methyl ester (7α, 11α, 17α) effective to contain an increase in the Flavivirus infection.

2. The method of claim 1 additionally comprising the step of administering intravenous fluid to the patient at one or both of: simultaneously with the administering to the patient the amount of pregn-4-ene-7,21-dicarboxylic acid, 9,11-epoxy-17-hydroxy-3-oxo, γ-lactone, methyl ester (7α, 11α, 17α) or following the administering of the amount of pregn-4-ene-7,21-dicarboxylic acid, 9,11-epoxy-17-hydroxy-3-oxo, γ-lactone, methyl ester (7α, 11α, 17α) to the patient.

3. The method of claim 1 additionally comprising the steps of:
determining that the patient has the Flavivirus infection prior to the administering to the patient of the pregn-4-ene-7,21-dicarboxylic acid, 9,11-epoxy-17-hydroxy-3-oxo, γ-lactone, methyl ester (7α, 11α, 17α); and
determining that the patient has contained the Flavivirus infection after the administering to the patient of pregn-4-ene-7,21-dicarboxylic acid, 9,11-epoxy-17-hydroxy-3-oxo, γ-lactone, methyl ester (7α, 11α, 17α).

4. The method of claim 3 wherein the pregn-4-ene-7,21-dicarboxylic acid, 9,11-epoxy-17-hydroxy-3-oxo, γ-lactone, methyl ester (7α, 11α, 17α) is administered via intra venous introduction.

5. The method of claim 3 wherein the pregn-4-ene-7,21-dicarboxylic acid, 9,11-epoxy-17-hydroxy-3-oxo, γ-lactone, methyl ester (7α, 11α, 17α) is administered via oral ingestion.

6. The method of claim 3 wherein the pregn-4-ene-7,21-dicarboxylic acid, 9,11-epoxy-17-hydroxy-3-oxo, γ-lactone, methyl ester (7α, 11α, 17α) is administered via transdermal patch.

7. The method of claim 1, wherein the pregn-4-ene-7,21-dicarboxylic acid, 9,11-epoxy-17-hydroxy-3-oxo, γ-lactone, methyl ester (7α, 11α, 17α) has demonstrated antiviral effect against Dengue virus covering each of strains DEN-1, DEN-2, DEN-3 and DEN-4.

8. The method of claim 7 wherein a fever of Dengue virus is selected from a group including classical dengue fever, dengue hemorrhagic fever syndrome, and dengue shock syndrome.

9. The method of claim 1, additionally comprising the step of co-administration of at least one agent selected from a group including: an antiviral agent, a vaccine, interferon and a therapeutic compound.

10. The method of claim 1, wherein the pregn-4-ene-7,21-dicarboxylic acid, 9,11-epoxy-17-hydroxy-3-oxo, γ-lactone, methyl ester (7α, 11α, 17α) is administered systemically via introduction into the patient's bloodstream.

11. The method of claim 3 wherein the pregn-4-ene-7,21-dicarboxylic acid, 9,11-epoxy-17-hydroxy-3-oxo, γ-lactone, methyl ester (7α, 11α, 17α) has demonstrated antiviral effect against Zika virus.

12. The method of claim 11, wherein the administering of the pregn-4-ene-7,21-dicarboxylic acid, 9,11-epoxy-17-hydroxy-3-oxo, γ-lactone, methyl ester (7α, 11α, 17α) is to treat a Zika virus infection in a human.

13. The method of claim 12 additionally comprising the steps of:
determining that the patient has the Zika virus infection prior to the administering to the patient of the pregn-4-ene-7,21-dicarboxylic acid, 9,11-epoxy-17-hydroxy-3-oxo, γ-lactone, methyl ester (7α, 11α, 17α); and
determining that the patient has contained the Zika virus infection after the administering to the patient of the pregn-4-ene-7,21-dicarboxylic acid, 9,11-epoxy-17-hydroxy-3-oxo, γ-lactone, methyl ester (7α, 11α, 17α).

14. A method of treating or preventing a Zika virus infection of a fetus by administering an antiviral dose of pregn-4-ene-7,21-dicarboxylic acid, 9,11-epoxy-17-hydroxy-3-oxo, γ-lactone, methyl ester (7α, 11α, 17α) to a pregnant mother of the fetus.

15. The method of claim 14 wherein the pregn-4-ene-7,21-dicarboxylic acid, 9,11-epoxy-17-hydroxy-3-oxo, γ-lactone, methyl ester (7α, 11α, 17α) is administered to the mother of the fetus via transdermal patch.

16. The method of claim 14 wherein the pregn-4-ene-7,21-dicarboxylic acid, 9,11-epoxy-17-hydroxy-3-oxo, γ-lactone, methyl ester (7α, 11α, 17α) is administered to the mother of the fetus via intra venous introduction.

17. The method of claim 14 wherein the pregn-4-ene-7,21-dicarboxylic acid, 9,11-epoxy-17-hydroxy-3-oxo, γ-lactone, methyl ester (7α, 11α, 17α) is administered to the mother of the fetus via oral ingestion.

18. The method of claim 17 additionally comprising the step of administering intravenous fluid to the mother of the fetus.

19. The method of claim 17 additionally comprising the step of administering an active agent to the mother of the fetus.

20. A compound for treatment of a Flavivirus infection, the compound comprising: pregn-4-ene-7,21-dicarboxylic acid, 9,11-epoxy-17-hydroxy-3-oxo, γ-lactone, methyl ester (7α, 11α, 17α) and one or more of: an antiviral agent, a vaccine, interferon and a therapeutic agent.
